SEQUENCE LISTING (14 sequences)

SEQ ID NO: 1 - DNA, 21 bp, Canis familiaris
tcaatcaggtgatggagaagg

SEQ ID NO: 2 - DNA, 20 bp, Canis familiaris
tgctcgaggagtttcttggt

SEQ ID NO: 3 - DNA, 20 bp, Canis familiaris
tccctggagaagagctacga

SEQ ID NO: 4 - DNA, 20 bp, Canis familiaris
cttctgcatcctgtcagcaa

SEQ ID NO: 5 - DNA, 20 bp, Canis familiaris
tggatgactgagtagctgaa

SEQ ID NO: 6 - DNA, 20 bp, Canis familiaris
ggcctactgacttcacttat

SEQ ID NO: 7 - DNA, 20 bp, Canis familiaris
tccaggaatcaggaccattt

SEQ ID NO: 8 - DNA, 20 bp, Canis familiaris
gattcctaggggcttgctct

SEQ ID NO: 9 - DNA, 23 bp, Canis familiaris
catctacactgacaactccatcc

SEQ ID NO: 10 - DNA, 22 bp, Canis familiaris
caggttccacttcagtttgttc

SEQ ID NO: 11 - DNA, 20 bp, Canis familiaris
cctacgtccagccatcattt

SEQ ID NO: 12 - DNA, 20 bp, Canis familiaris
cagttcctccttggggtttg

SEQ ID NO: 13 - DNA, 28 bp, Canis familiaris
actatgtatcacttgaggctctggtttc

SEQ ID NO: 14 - DNA, 27 bp, Canis familiaris
agtctggcttgattcatcttgtgtatg

(19) United States
(12) Patent Application Publication
(10) Pub. No.: US 2013/0338080 A1
(43) Pub. Date: Dec. 19, 2013

(54) COMPOSITIONS AND METHODS FOR TREATING AN ACTIVATED B-CELL DIFFUSE LARGE B-CELL LYMPHOMA

(71) Applicant: The Trustees of the University of Pennsylvania, Philadelphia, PA (US)

(72) Inventors: Nicola MASON, Philadelphia, PA (US); Michael May, Penn Valley, PA (US); Albert S. Baldwin, Chapel Hill, NC (US)

(73) Assignee: The Trustees of the University of Pennsylvania, Philadelphia, PA (US)

(21) Appl. No.: 13/899,128

(22) Filed: May 21, 2013

Related U.S. Application Data
(60) Provisional application No. 61/651,432, filed on May 24, 2012.

(51) Int. Cl. A61K 38/17; C07K 14/47; A61K 45/06

(52) U.S. Cl. CPC A61K 38/1709; A61K 45/06; C07K 14/47
USPC 514/19.3; 435/7.92; 435/6.12

(57) ABSTRACT

The invention relates to compositions and methods for treating an activated B-Cell Diffuse Large B-Cell Lymphoma (ABC-DLBCL). Specifically, the invention relates to treating ABC-DLBCL by administering an NF-κB Essential Modulator (NEMO) Binding Domain (NBD) peptide or a mimetic thereof that inhibits NF-κB activation.

### CROSS REFERENCE TO RELATED APPLICATIONS

This application claims priority to U.S. Provisional Patent Application 61/651,432, filed May 24, 2012, which is incorporated by reference herein in its entirety.

### GOVERNMENT INTEREST

The work described in this application was, in part, supported by the United States Department of Health and Human Services, the National Institutes of Health, Grant Number NIHUC2 CA 148149. United States Government may have certain rights in this application.

### FIELD OF THE INVENTION

The invention relates to compositions and methods for treating an activated B-Cell Diffuse Large B-Cell Lymphoma (ABC-DLBCL). Specifically, the invention relates to treating ABC-DLBCL by administering an NF-κB Essential Modulator (NEMO) Binding Domain (NBD) peptide or a mimetic thereof that inhibits NF-κB activation.

### BACKGROUND OF THE INVENTION

Diffuse Large B-Cell Lymphoma (DLBCL) is the most common adult lymphoid malignancy with approximately 30,000 new cases diagnosed each year in the United States. Gene expression profiling has revealed the presence of at least three subtypes of DLBCL, Activated B-Cell (ABC-DLBCL), Germinal Center B-Cell (GBC-DLBCL) and Primary Mediastinal B-Cell Lymphoma (PMBL), that have distinct molecular signatures and different clinical outcomes. ABC-DLBCL is the most aggressive subtype and is less responsive to conventional multi-agent chemotherapy than GCB-DLBCL and PMBL. Even with the addition of Rituximab, the overall survival for patients with ABC-DLBCL is 47%.

ABC-DLBCL is characterized by the presence of constitutive canonical NF-κB activity that drives expression of NF-κB target genes. In health, NF-κB activation is highly regulated. NF-κB dimers are held inactive in the cytoplasm through their association with inhibitory IκB family members. Engagement of cell surface receptors by many different infectious and inflammatory stimuli leads to phosphorylation and activation of the IKK complex, which phosphorylates IκBα and targets it for ubiquitination and proteosomal degradation. This allows NF-κB dimers to translocate to the nucleus where they bind to target gene promoters and enhancers and initiate transcription. Somatic activating mutations in genes encoding regulatory proteins upstream of the IKK complex have been identified in patients with ABC-DLBCL and lead to constitutive phosphorylation and activation of the IKK complex. Constitutive, canonical NF-κB activity is essential for ABC-DLBCL cell survival and inhibition of this pathway with small molecule inhibitors of IKK, IκBα super-repressors, proteosome inhibitors, or inhibitors upstream of the IKK complex promotes cell cycle arrest, chemotherapeutic sensitivity and apoptosis in ABC-DLBCL cell lines.

The high-quality draft genome sequence of the dog has revealed its close phylogenetic relationship with man, emphasizing the potential benefit of canine models in identifying disease genes and evaluating response to novel therapies. In particular, the dog is being increasingly recognized as a clinically relevant, spontaneous large animal model for human Non Hodgkin's Lymphoma (NHL). NHL is the most common, spontaneous, hematopoietic malignancy in dogs, with an annual incidence of 30/100,000. DLBCL is the most common subtype of canine NHL and shares similar biologic, behavioral, molecular and genetic characteristics with DLBCL in humans. Dogs with DLBCL are treated with the same cytotoxic agents used in human DLBCL patients that inhibit cell division and induce apoptosis. However, as in human patients, clinical remission is not maintained and 85-90% of canine patients relapse with lethal, drug-resistant lymphoma within 6 to 9 months of initial diagnosis and treatment.

Accordingly, there exists a need to develop compositions to treat ABC-DLBCL and improve treatment modalities.

### SUMMARY OF THE INVENTION

In one embodiment, the invention provides a method for treating an activated B-Cell Diffuse Large B-Cell Lymphoma (ABC-DLBCL) in a subject, the method comprising: administering to said subject a therapeutically effective amount of an NF-κB activation inhibitor, wherein said NF-κB activation inhibitor is an NF-κB Essential Modulator (NEMO) Binding Domain (NBD) peptide or a mimetic thereof, thereby treating said ABC-DLBCL in said subject.

In another embodiment, the invention provides a method for inhibiting the growth of a tumor associated with a relapsed, refractory (e.g., chemoresistant) large B-Cell lymphoma in a subject, the method comprising: administering to said subject a therapeutically effective amount of an NF-κB activation inhibitor, wherein said an NF-κB activation inhibitor is an NBD peptide or a mimetic thereof, thereby inhibiting the growth of said tumor associated with said relapsed, refractory large B-Cell lymphoma in said subject.

In another embodiment, the invention provides a pharmaceutical composition to treat an ABC-DLBCL in a subject, comprising: a therapeutically effective amount of an NF-κB activation inhibitor, wherein said an NF-κB activation inhibitor is an NBD peptide or a mimetic thereof, wherein said NF-κB activation inhibitor is present in an amount effective to treat said ABC-DLBCL.

In another embodiment, the invention provides a pharmaceutical composition to inhibit the growth of a tumor associated with a relapsed, refractory large B-Cell lymphoma in a subject, comprising: a therapeutically effective amount of an NF-κB activation inhibitor, wherein said NF-κB activation inhibitor is an NBD peptide or a mimetic thereof, and wherein said NF-κB activation inhibitor is present in an amount effective to inhibit the growth of said tumor associated with said relapsed, refractory large B-Cell lymphoma.

In another embodiment, the invention provides a method for enhancing a response to chemotherapy to treat an ABC-DLBCL in a subject, the method comprising: administering to said subject a therapeutically effective amount of an NF-κB activation inhibitor, wherein said NF-κB activation inhibitor is an NBD peptide or a mimetic thereof, thereby enhancing said response to chemotherapy to treat said ABC-DLBCL in said subject.

In another embodiment, the invention provides a method for treating an ABC-DLBCL in a subject, the method comprising: collecting a biological sample from said subject; determining the level of NF-κB activity in said sample; administering to said subject a therapeutically effective amount of an NF-κB activation inhibitor if the level of NF-κB activity is higher than a standard pre-determined level, wherein said NF-κB activation inhibitor is an NBD peptide or a mimetic thereof, thereby treating said ABC-DLBCL in said subject.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1. Constitutive canonical NF-κB activity in dogs with spontaneous DLBCL. (A) Whole cell protein extracts of normal lymph nodes (NLN) taken from 3 healthy dogs and lymph nodes taken from 4 dogs with primary, untreated DLBCL were evaluated for NF-κB DNA binding activity by EMSA. Results shown are representative of 13 dogs with DLBCL. (B) Whole cell protein extracts of lymph nodes taken from 2 dogs with primary, untreated DLBCL were subject to EMSA and supershift analysis with antibodies against p65, p50, c-Rel, or control rabbit IgG. Results shown are representative of 7 dogs with DLBCL. (C) Immunohistochemical staining for p65 was performed on lymph node sections from healthy dogs and dogs with DLBCL. Rabbit IgG was used as an isotype control. Arrowheads indicate nuclear p65 staining. Results shown are representative of 6 dogs with DLBCL and 4 healthy dogs (NLN). (D) Whole cell protein extracts of NLN and lymph nodes taken from 3 dogs with primary, untreated DLBCL were evaluated for the presence of p-IKKβ and p-IKBα by Western blot. Results shown are representative of 9 dogs with DLBCL. β-actin was used as an endogenous loading control.

FIG. 2. Expression of anti-apoptotic NF-κB target genes in canine DLBCL. RNA was isolated from lymph node biopsies of dogs with DLBCL at the time of diagnosis and prior to chemotherapy. qRT-PCR was performed for Bcl-2, c-FLIP and XIAP. Values are expressed as relative quantification using β-actin as an endogenous control. RNA isolated from biopsy samples of 3 healthy canine lymph nodes were used as a normalizing control. All data shown represent means+/−SEMs of reactions performed in triplicate. Statistical analysis was performed using a two-tailed Student's ‘t’ test where *p<0.05.

FIG. 3. NBD Peptide Inhibits Constitutive NF-κB Activity in Canine Primary DLBCL in vitro. Primary canine DLBCL cells were treated with either 100 μM NBD peptide or mutant peptide or with DMSO (vehicle control) for 12 hours. 25 μg of whole cell extract was immunoblotted using antibodies against (A) p-IKKβ and pan-IKKβ and (B) p-IκBα and pan-IκBα. β-actin was used as an endogenous loading control. Results are representative of 3 individual dogs. Canine PBMCs with or without TNF-a were included as positive and negative controls for p-IKKβ and p-IκBα. (C) Primary canine DLBCL cells were either untreated (grey histogram) or treated (open histograms) with either 100 μM NBD peptide or mutant peptide for 12 hours and then analyzed by flow cytometry for Annexin-V. Results are representative of 3 individual dogs. (D) Healthy canine PBMCs were treated with 100 μM NBD peptide or mutant peptide for 12 hours and then analyzed by flow cytometry for Annexin-V. Histograms are gated on CD21+ lymphocytes. (E) OCI-Ly10 and SUDHL-6 cells were treated with 0, 25 μM or 50 μM NBD peptide for 24 hours. Cell death was quantified by Trypan Blue. Data represent the percent increase in cell death in treated wells compared to untreated cells. Cell counts were performed in triplicate, and data shown represents means+/−SEMs. Statistical analysis was performed using a two-tailed Student's ‘t’ test. *p<0.05. Results are representative of 3 separate experiments.

FIG. 4. Schematic of Pilot Clinical Trial.

FIG. 5. In vivo administration of NBD peptide inhibits NF-κB target gene expression in malignant lymph nodes of dogs with relapsed/resistant DLBCL. (A; left panel) qRT-PCR of malignant lymph nodes of dogs with relapsed DLBCL at enrollment. Values are expressed as relative quantification using β-actin as an endogenous control. RNA isolated from biopsy samples of 3 healthy canine lymph nodes were used as a normalizing control. (A; right panel) qRT-PCR of malignant lymph nodes 24 hours post NBD peptide injection. RNA isolated from biopsy samples of the same dogs prior to NBD peptide treatment was used as a normalizing control. All data shown represents means+/−SEMs of reactions performed in triplicate. (B) Comparison of percent decrease in mass between NBD injected lymph node and contra-lateral, malignant, non-injected lymph node of 4 study dogs. Measurements were taken immediately prior to and one week post NBD peptide administration. (C) Percent decrease in mass of NBD injected lymph nodes from 2 dogs that received multi-nodal injections of NBD peptide.

### DETAILED DESCRIPTION OF THE INVENTION

The invention relates to compositions and methods for treating an activated B-Cell Diffuse Large B-Cell Lymphoma (ABC-DLBCL). Specifically, the invention relates to treating ABC-DLBCL by administering an NF-κB activation inhibitor, for example, an NF-κB Essential Modulator (NEMO) Binding Domain (NBD) peptide or a mimetic thereof.

In one embodiment, provided herein is a method for treating an ABC-DLBCL in a subject, the method comprising: administering to said subject a therapeutically effective amount of an NF-κB activation inhibitor, wherein said NF-κB activation inhibitor is an NBD peptide or a mimetic thereof, thereby treating said ABC-DLBCL in said subject. In another embodiment, provided herein is a method for inhibiting the growth of a tumor associated with a relapsed, refractory (e.g., chemoresistant) large B-Cell lymphoma in a subject, the method comprising: administering to said subject a therapeutically effective amount of an NF-κB activation inhibitor, wherein said an NF-κB activation inhibitor is an NBD peptide or a mimetic thereof, thereby inhibiting the growth of said tumor associated with said relapsed, refractory large B-Cell lymphoma in said subject.

In yet another embodiment, provided herein is a method for enhancing a response to chemotherapy to treat an ABC-DLBCL in a subject, the method comprising: administering to said subject a therapeutically effective amount of an NF-κB activation inhibitor, wherein said NF-κB activation inhibitor is an NBD peptide or a mimetic thereof, thereby enhancing said response to chemotherapy to treat said ABC-DLBCL in said subject.

Surprisingly and expectedly, the inventors of the instant application found that NBD peptide inhibits constitutive NF-κB activity and reduces tumor burden in a canine model of relapsed, refractory Diffuse Large B-Cell Lymphoma.

ABC-DLBCL is an aggressive, poorly chemoresponsive lymphoid malignancy characterized by constitutive canonical NF-κB activity that promotes lymphomagenesis and chemotherapy resistance via over-expression of anti-apoptotic NF-κB target genes. The inventors have investigated NF-κB as a therapeutic target using the dog as a clinically relevant, spontaneous, animal model of DLBCL. The inventors show that as in human ABC-DLBCL, malignant lymphocytes from dogs with DLBCL have constitutive canonical NF-κB activity and over-express anti-apoptotic genes. The inventors demonstrate that the selective IKK inhibitor, NBD peptide inhibits NF-κB activity in vitro and leads to apoptosis of malignant B cells. The inventors show in vivo that intra-tumoral injections of NBD peptide inhibit NF-κB gene expression and reduce tumor burden in dogs with DLBCL. These studies show the therapeutic relevance of NF-κB inhibition in vivo in a canine DLBCL model and thereby show the treatment of human ABC-DLBCL.

As used herein, “NF-κB activation inhibitor” may refer to any molecule that is able to directly or indirectly inhibit the activity of NF-κB.

NF-κB activation inhibitors are well known in the art. In an exemplary embodiment, NF-κB activation inhibitor is an NBD peptide or a mimetic thereof. NBD peptide and its mimetic molecules are well known in the art and fully described in US Patent Application Publications US 2007-0225315, US 2006-0293244, US 2003-0104622, US 2003-0219826, US 2005-0074884, US 2009-0075902, and US 20120101028; U.S. Pat. No. 6,864,355, U.S. Pat. No. 7,049,395, U.S. Pat. No. 6,881,825; and International Patent Application PCT/US03/04632, all of which are incorporated by reference herein in their entirety.

In one embodiment, NBD peptide is a selective inhibitor of the IKK complex that consists of 11 amino acids located at the carboxy terminus of the catalytic IKKβ subunit that binds to the scaffold protein NF-κB Essential Modulator (NEMO). The core of six amino acids in the NBD can also present in IKKα and facilitates its association with NEMO. In some embodiments, NBD peptide inhibits the interaction of both IKKα and IKKβ with NEMO and prevents assembly of the IKK complex. Fusion of NBD peptide to protein transduction domains, such as that within the drosophila antennapedia protein enables it to enter cells and effectively inhibit canonical NF-κB activation in response to TNFα, lipopolysaccharide and Toll-Like Receptor ligation. See May et al., Science. 2000 Sep. 1; 289(5484):1550-4; May et al. J Biol Chem. 2002 Nov. 29; 277(48):45992-6000; Solt et al., J Biol Chem. 2009 Oct. 2; 284(40):27596-608; Orange et al., Mol Life Sci. 2008 November; 65(22):3564-91, all of which are incorporated by reference herein in their entirety.

As used herein, the term “NEMO Binding Domain” or “NBD” includes any domain capable of binding to NEMO at the region where NEMO usually interacts with an IKK (e.g., IKKα and/or IKKβ).

The amino acid and nucleic acid sequences of NBD peptide are well known in the art and available in public databases. The amino acid or nucleic acid sequence described herein includes a homologue, a variant, an isomer, or a functional fragment thereof. Each possibility is a separate embodiment of the invention.

The term “mimetic,” as used herein, includes molecules which mimic the chemical structure of a peptidic structure and retain the functional properties of the peptidic structure. Approaches to designing peptide analogs, derivatives and mimetics are well known in the art. See e.g., Farmer, P. S. in Drug Design (E. J. Ariens, ed.) Academic Press, New York, 1980, vol. 10, pp. 119-143; Ball. J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270. See also Sawyer, T. K. (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117:11113-11123; Smith, A. B. 3rd, et al. (1994) J. Am. Chem. Soc. 116:9947-9962; and Hirschman, R., et al. (1993) J. Am. Chem. Soc. 115:12550-12568.

Examples of peptidomimetics include peptidic compounds in which the peptide backbone is substituted with one or more benzodiazepine molecules. See e.g., James, G. L. et al. (1993) Science 260:1937-1942. The term “peptidomimetic,” as used herein, includes isosteres. The term “isostere,” as used herein, includes a chemical structure that can be substituted for a second chemical structure because the steric conformation of the first structure fits a binding site specific for the second structure. The term also includes peptide back-bone modifications (e.g., amide bond mimetics) well known to those skilled in the art. Such modifications include, for example, but are not limited to, modifications of the amide nitrogen, the a-carbon, amide carbonyl, complete replacement of the amide bond, extensions, deletions or backbone crosslinks.

NBD peptide also includes its derivatives. Examples of peptide derivatives include peptides in which an amino acid side chain, the peptide backbone, or the amino- or carboxy-terminus has been derivatized (e.g., peptidic compounds with methylated amide linkages).

Methods for making NF-κB activation inhibitors are also well known in the art. Any suitable method, known to a person of skilled in the art, can be used to make NF-κB activation inhibitors.

In another embodiment, provided herein is a pharmaceutical composition to treat an ABC-DLBCL in a subject, comprising: a therapeutically effective amount of an NF-κB activation inhibitor, wherein said an NF-κB activation inhibitor is an NBD peptide or a mimetic thereof, wherein said NF-κB activation inhibitor is present in an amount effective to treat said ABC-DLBCL. In yet another embodiment, provided herein is a pharmaceutical composition to inhibit the growth of a tumor associated with a relapsed, refractory (e.g., chemoresistant) large B-Cell lymphoma in a subject, comprising: a therapeutically effective amount of an NF-κB activation inhibitor, wherein said NF-κB activation inhibitor is an NBD peptide or a mimetic thereof, and wherein said NF-κB activation inhibitor is present in an amount effective to inhibit the growth of said tumor associated with said relapsed, refractory large B-Cell lymphoma.

The invention also provides a pharmaceutical composition comprising an NF-κB activation inhibitor of the invention and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or additional therapeutic agents.

Pharmaceutically acceptable carriers include solvents, dispersion media, buffers, coatings, antibacterial and antifungal agents, wetting agents, preservatives, buggers, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof. Antibacterial and antifungal agents include parabens, chlorobutanol, phenol, ascorbic acid, and thimerosal.

The pharmaceutical compositions of the invention may be formulated in a variety of ways, including for example, liquid, semi-solid and solid dosage forms, such as liquid solutions (e.g., injectable and infusible solutions), dispersions or suspensions, tablets, pills, powders, liposomes and suppositories. In some embodiments, the compositions are in the form of injectable or infusible solutions. The composition is in a form suitable for oral, intravenous, intraarterial, intramuscular, subcutaneous, parenteral, transmucosal, transdermal, or topical administration. The composition may be formulated as an immediate, controlled, extended or delayed release composition.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the composition includes isotonic agents, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

Sterile injectable solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in US Appl. Publ. No. 2002/0102208 A1, which is incorporated herein by reference in its entirety.

Effective doses of the compositions of the present invention, for treatment of conditions or diseases as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

The pharmaceutical compositions of the invention may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

The invention further provides a composition or kit comprising a therapeutically effective amount of a NF-κB activation inhibitor, a chemotherapy agent, or combination thereof.

The invention further provides methods of treating a disease or condition, comprising administering to a mammal in need thereof a therapeutically effective amount of a NF-κB activation inhibitor, a chemotherapy agent, or combination thereof.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already with the disease or condition as well as those prone to having the disease or condition or those in which the disease or condition is to be prevented.

Examples of cancers/tumors which can be treated include an activated B-Cell Diffuse Large B-Cell Lymphoma or a cancer associated with the NF-κB activity. In a particular embodiment, cancers/tumor which can be treated is relapsed, refractory diffuse large B-cell lymphoma.

Methods of treating cancer include, e.g., inhibiting angiogenesis in the tumor, inhibiting tumor growth, inhibiting tumor migration, inhibiting proliferation or inhibiting invasion of tumor cells.

Cancers that express or overexpress or are associated with the expression or overexpression of NF-κB may be treated by the invention.

Cancers to be treated include primary tumors and secondary or metastatic tumors, as well as recurrent or refractory tumors. Recurrent tumors encompass tumors that appear to be inhibited by treatment with such agents, but recur up to five years, sometimes up to ten years or longer after treatment is discontinued. Refractory tumors are tumors that have failed to respond or are resistant to treatment with one or more conventional therapies for the particular tumor type. Refractory tumors include those that are hormone-refractory; those that are refractory to treatment with one or more chemotherapeutic agents; those that are refractory to radiation; and those that are refractory to combinations of chemotherapy and radiation, chemotherapy and hormone therapy, or hormone therapy and radiation

Therapy may be “first-line”, i.e., as an initial treatment in patients who have had no prior anti-cancer treatments, either alone or in combination with other treatments; or “second-line”, as a treatment in patients who have had one prior anti-cancer treatment regimen, either alone or in combination with other treatments; or as “third-line”, “fourth-line”, etc. treatments, either alone or in combination with other treatments.

Therapy may also be given to patients who have had previous treatments which have been partially successful but are intolerant to the particular treatment. Therapy may also be given as an adjuvant treatment, i.e., to prevent reoccurrence of cancer in patients with no currently detectable disease or after surgical removal of tumor.

More than one NF-κB activation inhibitors may be administered, either incorporated into the same composition or administered as separate compositions.

The NF-κB activation inhibitor may be administered alone, or in combination with one or more therapeutically effective agents or treatments. The other therapeutically effective agent may be conjugated to the NF-κB activation inhibitor, incorporated into the same composition as the NF-κB activation inhibitor, or may be administered as a separate composition. The other therapeutically agent or treatment may be administered prior to, during and/or after the administration of the NF-κB activation inhibitor.

In one embodiment, NF-κB activation inhibitor is co-administered with a chemotherapy agent. In another embodiment, NF-κB activation inhibitor is administered independently from an administration of a chemotherapy agent. In one embodiment, NF-κB activation inhibitor is administered first, followed by the administration of a chemotherapy agent. In another embodiment, a chemotherapy agent is administered first, followed by the administration of NF-κB activation inhibitor.

Other therapeutically effective agents/treatments include surgery, anti-neoplastics (including chemotherapeutic agents and radiation), anti-angiogenesis agents, antibodies to other targets, small molecules, photodynamic therapy, immunotherapy, cytotoxic agents, cytokines, chemokines, growth inhibitory agents, anti-hormonal agents, kinase inhibitors, cardioprotectants, immunostimulatory agents, immunosuppressive agents, agents that promote proliferation of hematological cells, and protein tyrosine kinase (PTK) inhibitors.

A chemotherapeutic agent may be administered as a prodrug. The term “prodrug” refers to a precursor or derivative form of a pharmaceutically active substance that is less cytotoxic to tumor cells compared to the parent drug and is capable of being enzymatically activated or converted into the more active parent form. The prodrugs that may find use with the compositions and methods as provided herein include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug.

The administration of the NF-κB activation inhibitor with other agents (e.g., chemotherapy agent) and/or treatments may occur simultaneously, or separately, via the same or different route, at the same or different times. Dosage regimens may be adjusted to provide the optimum desired response (e.g., a therapeutic or prophylactic response).

In one example, a single bolus may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the invention are dictated by and directly dependent on the unique characteristics of the active compound and the particular therapeutic or prophylactic effect to be achieved.

The composition of the invention may be administered only once, or it may be administered multiple times. For multiple dosages, the composition may be, for example, administered three times a day, twice a day, once a day, once every two days, twice a week, weekly, once every two weeks, or monthly.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

“Administration” to a subject is not limited to any particular delivery system and may include, without limitation, parenteral (including subcutaneous, intravenous, intramedullary, intraarticular, intramuscular, or intraperitoneal injection) rectal, topical, transdermal or oral (for example, in capsules, suspensions or tablets). Administration to a host may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, for example, Remington's Pharmaceutical Sciences, Mack Publishing Co.).

The methods of treatment described herein can be used to treat any suitable mammal, including primates, such as monkeys and humans, horses, cows, cats, dogs, rabbits, and rodents such as rats and mice. In one embodiment, the mammal to be treated is human.

Any patent, patent application publication, or scientific publication, cited herein, is incorporated by reference herein in its entirety.

The following examples are presented in order to more fully illustrate the preferred embodiments of the invention. They should in no way be construed, however, as limiting the broad scope of the invention.

### EXAMPLES

### Materials and Methods

### Cells and Reagents

Canine lymphocytes were cultured in complete RPMI media containing 10% FBS. The human GCB-DLBCL cell line SUDHL-6 was maintained in complete RPMI media, and the human ABC-DLBCL cell line OCI-Ly10 was maintained in complete Iscove's modified essential medium (IMDM) containing 20% FBS. Cell lines were obtained from Dr. Anne Novak (Mayo Clinic Cancer Center, Rochester, Mn). Malignant lymph node samples were collected from dogs with either primary or relapsed DLBCL following approval from the University of Pennsylvania's Institutional Animal Care and Use Committee. The histopathological diagnosis of DLBCL and the cytological diagnosis of large B cell lymphoma were made by board certified veterinary pathologists and by a board certified clinical pathologists respectively. NBD peptide was synthesized and purified by Dr. James I. Elliott (at the Howard Hughes Medical Institute Biopolymer-Keck Foundation Biotechnology Resource Laboratory, Yale University, New Haven, Conn.) using standard tertbutoxycarbonyl (t-Boc) chemistry, cleavage with hydrofluoric acid, and purification by reversed-phase HPLC (26). TNFα was purchased from Sigma-Aldrich (Saint Louis, Mo.).

### Western Blot Analyses

Cells were lysed in 50 mM Tris-HCL containing 1% NP-40, 150 mM NaCl, 2.5 mM EDTA, 5% glycerol, a 1:50 dilution of Protease Inhibitor Cocktail and 1:50 dilutions of Phosphatase Inhibitor Cocktails 2 and 3 (Sigma-Aldrich). Protein concentrations were determined by micro BCA assay (Thermo Scientific, Rockford, Ill.). Proteins were separated by SDS-PAGE (10%) and transferred to PVDF membrane. Membranes were probed with polyclonal rabbit anti-human antibodies against phospho-IKKα/β (16A6), pan IKKβ (L570), phospho-IκBα (14D4) and β-actin (4967) (Cell Signaling, Danvers, Mass.) or rabbit anti-human pan IκBα (C-21) (SantaCruz Biotechnology, Santa Cruz). An HRP-conjugated donkey anti-rabbit IgG was used as the secondary detection antibody (Amersham, Piscataway, N.J.). Blots were developed using ECL Plus (Amersham, Piscataway, N.J.) or SuperSignal West Femto (Thermo Scientific, Rockford, Ill.).

### Electrophoretic Mobility Shift Assay (EMSA)

Whole cell extracts of malignant canine lymphocytes were prepared as previously described. EMSAs were performed using 5 μg protein extract and a palindromic NF-κB binding sequence probe (Santa Cruz Biotechnology) as previously described. EMSA supershift assays were performed using 10 μg of protein extract and polyclonal rabbit anti-human p65 (sc-109×), p50 (sc-114×) and c-Rel (sc-70×) antibodies or control rabbit IgG (sc-2027) (Santa Cruz Biotechnology).

Quantitative Reverse Transcription PCR (qRT-PCR)

Total RNA extraction was performed using the RNeasy Mini Kit (Qiagen, Valencia, Calif.). Reverse transcription was performed using random hexamers and Superscript II reverse transcriptase (Invitrogen Corp., Carlsbad. Calif., USA). Transcript sequences for canine A1, c-FLIP, Cyclin D1 and IκBα were obtained from the NCBI (http://www.ncbi.nih.gov/Genbank) and were analyzed for secondary DNA structure using M-Fold (http://frontend.bioinfo.rpi.edu/applications/mfold/cgi-bin/dna-form1.cgi). Primers were designed using Primer 3 software (http://frodo.wi.mit.edu/primer3/) with the maximum self-complementarity score set at 5 and the maximum 3 self-complementarity score set to 0 to minimize primer-dimer formation (Table 1). Primer sequences for canine Bcl-2, XIAP and β-actin have been previously described. Quantitative RT-PCR was performed using SYBR Green (Fermentas, Glen Burnie, Md.). Samples were run in triplicate using standard conditions on an ABI 7500 sequence detector (Applied Biosystems, Carlsbad, Calif.) and data were analyzed using β-actin as an endogenous control. Dissociation curves were performed after each experiment to confirm the specificity of product amplification.

TABLE 1
Primer sequences for NF-κB target gene expression
analyses

| Gene | | RT-PCR primers | Accession # |
|---|---|---|---|
| A1 | Sense: | 5′-TCA ATC AGG TGA TGG AGA AGG-3′ | XM_545888 |
| | Antisense: | 5′-TGC TCG AGG AGT TTC TTG GT-3′ | |
| Actin | Sense: | 5′-TCC CTG GAG AAG AGC TAC GA-3′ | AF021873 |
| | Antisense: | 5′-CTT CTG CAT CCT GTC AGC AA-3′ | |
| Bcl-2 | Sense: | 5′-TGG ATG ACT GAG TAG CTG AA-3′ | NM_001002949 |
| | Antisense: | 5′-GGC CTA CTG ACT TCA CTT AT-3′ | |
| c-FLIP | Sense: | 5′-TCC AGG AAT CAG GAC CAT TT-3′ | XM_646682 |
| | Antisense: | 5′-GAT TCC TAG GGG CTT GCT CT-3′ | |
| Cyclin | Sense: | 5′-CAT CTA CAC TGA CAA CTC CAT CC-3′ | NM_001005757 |
| D1 | Antisense: | 5′-CAG GTT CCA CTT CAG TTT GTT C-3′ | |
| IκBα | Sense: | 5′-CCT ACG TCC AGC CAT CAT TT-3′ | XM_637413 |
| | Antisense: | 5′-CAG TTC CTC CTT GGG GTT TG-3′ | |
| XIAP | Sense: | 5′-ACT ATG TAT CAC TTG AGG CTC TGG TTT C-3′ | AY603038 |
| | Antisense: | 5′-AGT CTG GCT TGA TTC ATC TTG TGT ATG-3′ | |

### Immunohistochemistry

Immunohistochemical analysis was performed on formalin fixed, paraffin-embedded tissue sections of lymph nodes from healthy dogs and dogs with DLBCL. Sections were deparaffinized in xylene, rehydrated and boiled in sodium citrate buffer to unmask antigens. Endogenous peroxidase was blocked with 3% hydrogen peroxide. Immunohistochemistry was performed using a rabbit anti-human p65 antibody (C22B4) (Cell Signaling, Danvers, Mass.) or rabbit isotype control (sc-2027) (Santa Cruz Biotechnology) and the Vectastain Elite ABC Kit (Vector Laboratories, Burlingame, Calif.). Sections were developed using 3′,3′ diaminobinzidine (DAB) and counterstained with hematoxylin. Slides were viewed using a Nikon E600 infinity corrected upright microscope. Bright field images were acquired using a Nikon Digital Sight DS-Fi1 color camera using NIS-Element BR3.0 for image analysis.

### In Vitro NBD Peptide Treatment of Malignant B Lymphocytes

Freshly isolated or cryopreserved malignant lymphocytes from lymph nodes of dogs with DLBCL were seeded at 4×10⁶ cells/ml and treated with 100 μm of either NBD or mutant peptide in DMSO. After 12 h, whole cell extracts were evaluated by immunoblot for canonical NF-κB activity as described above, and for apoptosis by flow cytometry. The human ABC- and GCB-DLBCL cell lines OCI-Ly10 and SUDHL-6, were seeded at 0.5×10⁶ cells/ml and treated with 0, 25 μM or 50 μM NBD peptide. After 24 hours, cell death was evaluated by Trypan Blue.

### Flow Cytometry

Detection of apoptotic cell death was performed using the PE Annexin-V Apoptosis Detection Kit (BD Biosciences, San Jose, Calif.). Cells were acquired on a FACSCalibur cytometer (BD Biosciences) and analyzed using TreeStar FlowJo software. Immunophenotyping of primary lymphoma cells was performed using the following antibodies: FITC conjugated rat anti-canine CD3 (Serotec, Raleigh, N.C.), APC conjugated mouse anti-human CD79a (BD Pharmingen, San Diego, Calif.) and mouse anti-canine CD21-like molecule (Serotec, Raleigh, N.C.). A FITC-labeled goat anti-mouse IgG secondary antibody was used to detect anti-CD21 antibody. For CD79a staining, cells were fixed with 1% paraformaldehyde and then permeabilized with 0.1% saponin prior to staining.

### Pilot Trial

All in vivo studies were performed following approval from the University of Pennsylvania's Institutional Animal Care and Use Committee and the University of Pennsylvania School of Veterinary Medicine Clinical Review Board. Client-owned dogs were enrolled with the following inclusion criteria: 1) cytological confirmation of relapsed large B-cell lymphoma, 2) stage III-V, substage a, with at least 1 set of bilateral, enlarged, measurable, malignant lymph nodes, 3) life expectancy of greater than 1 month, 4) no concurrent systemic disease, 5) adequate hematologic, renal and hepatic function and 6) the presence of constitutive NF-κB activity within malignant lymph nodes as determined by EMSA. All dogs enrolled in the pilot trial had been previously treated with a range of rescue chemotherapeutic agents including L-asparaginase, cyclophosphamide, vincristine, prednisone, lomustine, doxorubicin, mechlorethamine, procarbazine, dacarbazine and total body irradiation. Signed informed consent was obtained from all owners before entry into the study. Baseline evaluation included a complete medical history, full clinical examination, complete blood count (CBC), chemistry screen (CS) and urinalysis, malignant lymph node cytology and flow cytometric immunophenotyping using a basic panel of antibodies including CD3, CD21 and CD79a. Eligible dogs received either 5 or 10 mg NBD peptide/100 g malignant lymph node mass. Node mass was calculated based on two dimension caliper measurements which were averaged and considered the diameter of the lymph node sphere. Volume was determined based on V=⅔πr³. Based on our previous assessments, 1 cm³ of malignant lymph node mass is equivalent to ˜379 mg. Peptide was injected directly into one malignant node and the same node was biopsied 24 hours later. qRT-PCR for NF-κB target gene expression was performed on node tissue taken 24 hours post NBD peptide treatment and was normalized to values obtained pre NBD peptide. No control dogs were included in this pilot study. Following the post treatment biopsy, patients received rescue chemotherapy as determined by their primary attending oncologist. All dogs received a follow up full clinical examination plus CBC, CS and urinalysis one-week post NBD peptide injection. Mass of the NBD treated lymph node and the contra-lateral, malignant, untreated lymph node was calculated before, and one week post NBD peptide administration.

### Example 1

### Constitutive Canonical NF-κB Activity Occurs in Dogs with DLBCL

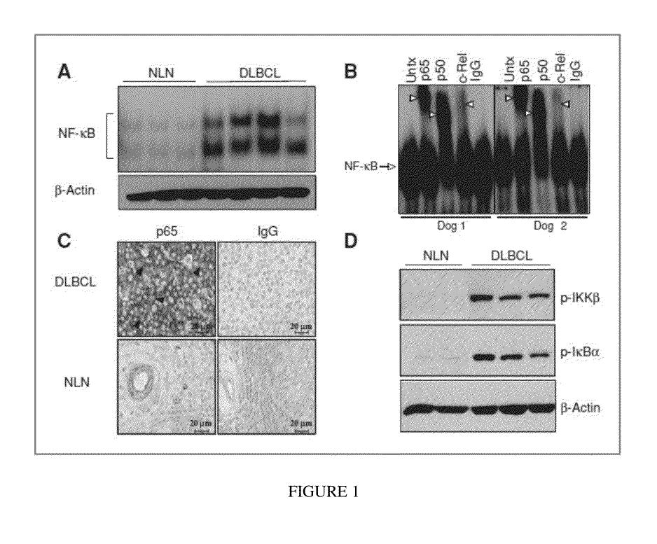

To determine whether constitutive NF-κB activity is present in dogs with spontaneous, histologically confirmed DLBCL, EMSA was performed on biopsy samples taken from the peripheral lymph nodes of 13 dogs with DLBCL at the time of diagnosis and prior to chemotherapy. Constitutive NF-κB activity was detected in all 13 samples. In contrast, NF-κB activity was not detected in normal lymph nodes from healthy dogs (FIG. 1A). To determine whether constitutive NF-κB activity was associated with the canonical pathway, malignant lymph node tissue from 11 dogs with newly diagnosed, histologically confirmed DLBCL was analyzed by EMSA supershift using antibodies directed against p50, p65 and c-Rel. In 7 dogs, p50, p65 and c-Rel were all present in the active NF-κB complexes (FIG. 1B). In 2 dogs only p50 and p65 were present, and in 2 dogs only p50 was identified (data not shown). To further confirm the presence of constitutive canonical NF-κB activity, formalin-fixed, paraffin-embedded lymph node tissues from 6 dogs with DLBCL were evaluated for the presence of nuclear p65 by immunohistochemistry (FIG. 1C). All tissue sections had abundant cytoplasmic and nuclear-localized p65 whereas 4 healthy canine lymph node sections had minimal p65 staining.

To determine whether the detected canonical NF-κB activity was associated with constitutive IKK activity, the phosphorylation status of the IKK complex and the inhibitory IκBα protein were evaluated by immunoblot (FIG. 1D). p-IKKβ and p-IκBα were detected in 8 out of 9 lymph node biopsy samples taken from dogs with histologically confirmed, untreated DLBCL with constitutively active NF-κB as determined by EMSA. In contrast, p-IKKβ and p-IκBα were only detected at very low levels in normal lymph nodes taken from healthy dogs (FIG. 1D). Taken together, these findings demonstrate that constitutive canonical NF-κB activity occurs in the malignant tissue of dogs with DLBCL, and that in the majority of dogs this activity is associated with IKK activation and downstream IκBα phosphorylation. These data parallel findings in human ABC-DLBCL, and suggest that dogs with DLBCL represent a clinically relevant, spontaneous large animal model in which to evaluate the therapeutic effects of NF-κB inhibition in the treatment of ABC-DLBCL.

### Example 2

### Anti-Apoptotic NF-κB Target Genes are Up-Regulated in Canine DLBCL

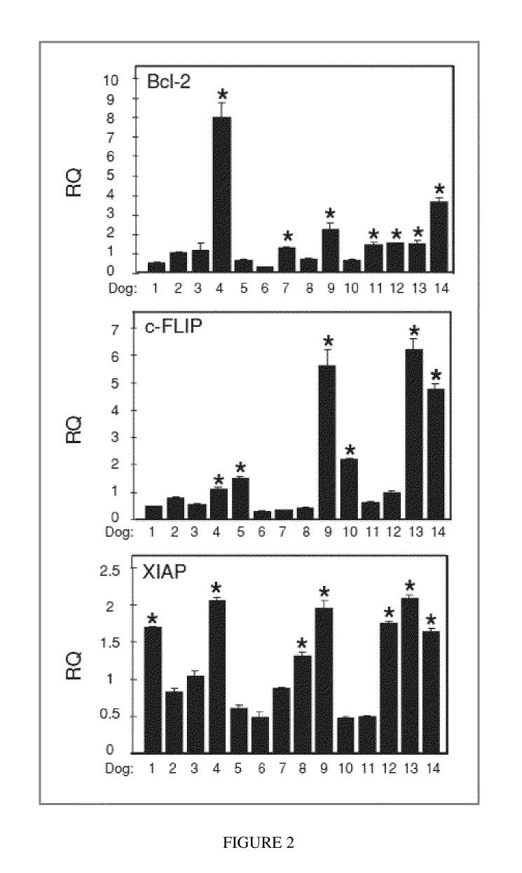

To determine whether NF-κB target genes are up-regulated in canine DLBCL, malignant lymph node tissue from 14 dogs with histologically confirmed DLBCL and constitutive NF-κB activity was evaluated by qRT-PCR for Bcl-2, c-FLIP and XIAP gene expression (FIG. 2). Tissue samples were obtained at the time of diagnosis and prior to chemotherapy. Using healthy canine lymph node tissue as a normalizing control, statistically significant increases in Bcl-2 gene expression were identified in 7 dogs. In addition, c-FLIP and XIAP gene expression was significantly increased in 6 and 7 dogs, respectively. These results suggest that constitutive canonical NF-κB activity in canine DLBCL promotes the over-expression of anti-apoptotic NF-κB target genes. This finding parallels that in human patients with ABC-DLBCL and indicates that constitutive canonical NF-κB activity in the dog may contribute to lymphomagenesis and chemoresistance.

### Example 3

### NBD Peptide Inhibits Constitutive NF-κB Activity and Promotes Apoptosis in DLBCL Cells In Vitro

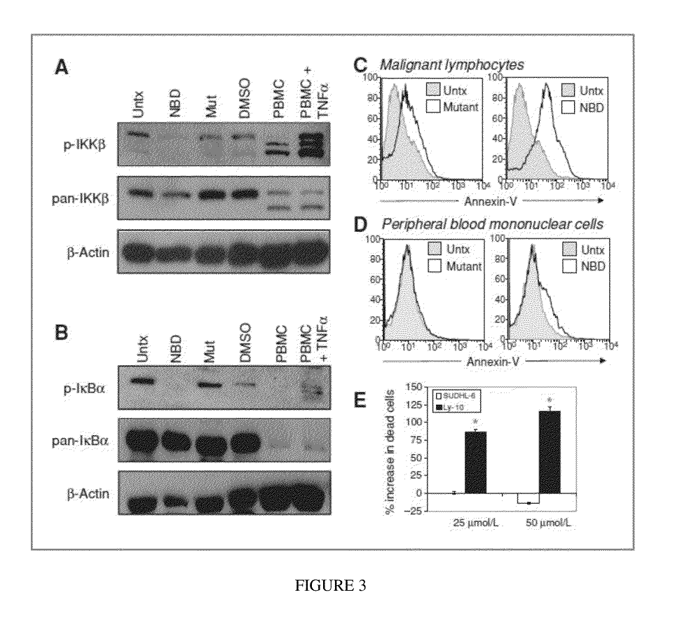

To determine whether NBD peptide can inhibit constitutive phosphorylation of IKK and prevent downstream IκBα phosphorylation, cells from malignant lymph nodes of dogs with DLBCL were treated in vitro with either 100 μM NBD peptide or 100 μM mutant peptide for 12 hours and then analyzed for the presence of p-IKKβ and p-IκBα by immunoblot (FIGS. 3A and B). Cells treated with NBD peptide showed a marked reduction of p-IKKβ compared to untreated cells, and those treated with either mutant peptide or DMSO vehicle control. NBD peptide treatment also resulted in complete inhibition of IκBα phosphorylation (FIG. 3B). In contrast, treatment with mutant peptide or DMSO (vehicle control) did not inhibit IκBα phosphorylation.

To determine the effects of NF-κB inhibition on DLBCL cell apoptosis, malignant lymphocytes harvested from canine DLBCL tumors were treated in vitro with NBD or mutant peptide and analyzed by flow cytometry (FIG. 3C). After 12 hours incubation with NBD peptide, more than 50% of DLBCL cells stained positive for Annexin-V while only 15% of cells treated with mutant peptide appeared apoptotic. Comparable results were identified in malignant cells taken from the lymph nodes of 3 dogs with DLBCL. In contrast, when PBMCs isolated from healthy dogs were treated with either 100 μM NBD or mutant peptide for 12 hours, less than 5% of PBMCs stained positive for Annexin-V (FIG. 3D). Collectively, these data show that NBD peptide inhibits constitutive NF-κB activity in canine DLBCL cells, leading to selective, rapid apoptosis of malignant cells.

To confirm a selective cytotoxic effect of NBD peptide on human ABC-DLBCL cells, OCl-Ly10 cells and the GCB-DLBCL cell line SUDHL-6 were treated with NBD peptide for 24 hours and the percentage increase in cell death compared to untreated cells was calculated. At 24 hours, NBD peptide induced a dose dependent increase in cell death of the ABC-DLBCL cell line, OCl-Ly10. In contrast, NBD peptide did not induce cell death in the GCB-DLBCL cell line at either concentration used (FIG. 3E).

### Example 4

### In Vivo Administration of NBD Peptide Inhibits NF-κB Target Gene Expression and Leads to a Reduction in Tumor Burden in Dogs with Relapsed, Chemoresistant Large B-Cell Lymphoma

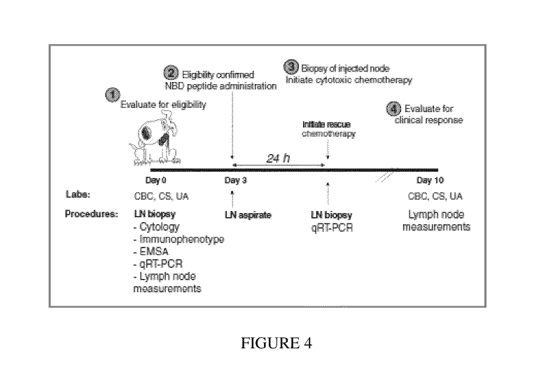

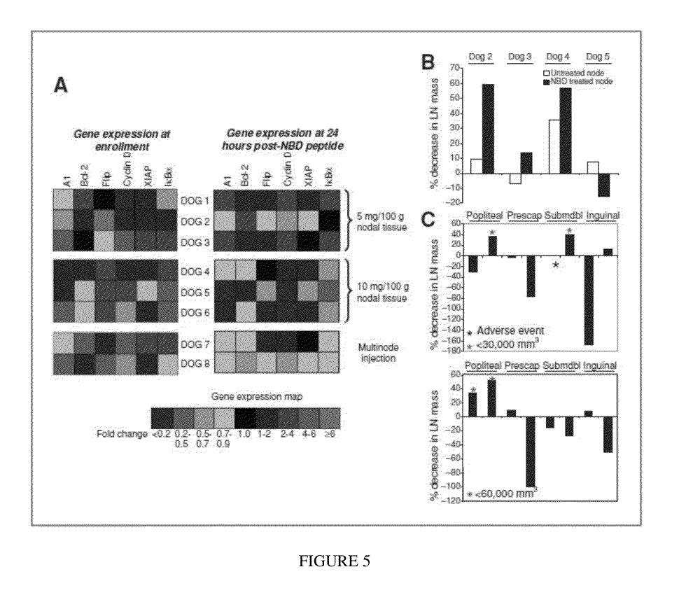

To determine whether NBD peptide can inhibit NF-κB target gene expression in malignant lymphoid tissue in vivo, privately owned dogs with relapsed, chemoresistant large B-cell lymphoma were enrolled to a small pilot study (FIG. 4). Eligibility criteria included the presence of histologically or cytologically confirmed large B-cell lymphoma and constitutive NF-κB activity in malignant lymph nodes as determined by EMSA. Response to NBD peptide was determined by evaluating NF-κB target gene expression pre and 24 hours post NBD peptide administration. Clinical response to NBD peptide was determined by comparing the mass of the injected lymph node before NBD peptide and 1 week after administration of peptide plus rescue chemotherapy. The mass of the contra-lateral malignant lymph node that was not injected with NBD peptide but was exposed to systemic rescue chemotherapy was also determined at both time points and the change in lymph node mass was calculated for both nodes and compared. Six eligible dogs were enrolled to the study, three dogs received 5 mg NBD peptide/100 g of node tissue and three received 10 mg NBD peptide/100 g node tissue via a single intra-nodal injection. At the time of enrollment, qRT-PCR analysis of lymph node tissue revealed that all dogs over-expressed at least 2 out of 6 NF-κB target genes that promote cellular proliferation and inhibit apoptosis when compared to normal lymph node tissue (FIG. 5A left panel). 24 hours post NBD peptide administration 1 out of 3 dogs receiving 5 mg NBD peptide/100 g nodal tissue showed a reduction in the expression of 4/6 NF-κB target genes within the malignant node when compared to pre-NBD treatment gene expression values. At the higher dose of NBD peptide all three dogs showed a reduction in the expression of at least 3/6 NF-κB target genes including Bcl-2, Cyclin D1 and IκBα when compared to pre-NBD treatment values. (FIG. 5A right panel).

Response to NBD peptide was determined by comparing the change in mass of the NBD peptide treated lymph node with change in mass of the contra-lateral, malignant, non-injected lymph node. Results were available for 4 of the 6 treated dogs (FIG. 5B). Rescue chemotherapy protocols administered to each dog after NBD peptide treatment varied amongst dogs and were determined by the referring oncologist. In 3 out of 4 dogs, the NBD peptide treated lymph node showed between a 1.5 and 6 fold reduction in mass when compared to the contra-lateral, malignant lymph node that did not receive NBD peptide. Two dogs (dogs 7 and 8) were treated on a compassionate basis and given intranodal injections of 10 mg NBD/100 g tumor into 8 peripheral enlarged lymph nodes, resulting in cumulative NBD doses of 0.33 and 0.8 mg/kg body weight respectively. This amendment to the original protocol was approved by the University of Pennsylvania's Institutional Animal Care and Use Committee. At enrollment, each dog over-expressed at least 3 NF-κB target genes (FIG. 5A left panel). 24 hours after NBD peptide administration, reductions in NF-κB target gene expression were observed in 3/6 and 6/6 evaluated genes, respectively (FIG. 5A right panel). In these two dogs, a reduction in lymph node mass was observed in 3/8 and 4/8 NBD treated lymph nodes (FIG. 5C). Despite NBD peptide and systemic chemotherapy, the remaining lymph nodes continued to increase in size. In both dogs, the lymph nodes that showed a reduction in mass following NBD peptide treatment were the smallest nodes at the time of treatment.

In this study we first set out to determine whether dogs with DLBCL represent an appropriate, spontaneous, large animal model in which to evaluate the therapeutic effects of NF-κB inhibition. DLBCL in the dog is often characterized by total effacement of lymph node architecture with large malignant B-cells that have a high mitotic index and multiple, prominent nucleoli. This malignancy in dogs is frequently associated with over-expression of c-myc that occurs as a result of chromosomal translocations that place the oncogene under control of the IgH promoter. While these histocytological and cytogenetic changes are also found in ABC-DLBCL in humans, it is unknown whether canine DLBCL is characterized by constitutive canonical NF-κB activity. Here we show that dogs with histologically confirmed DLBCL have constitutive canonical NF-κB signaling in malignant lymphoid tissue at the time of diagnosis. In most cases evaluated, p50/p65 and p50/c-Rel heterodimers were present in the activated complex. Only 2 biopsy samples had evidence of active p50 in the apparent absence of active p65 or c-Rel suggesting the presence of p50 homodimers or complexes with RelB in these patients. These differences may be attributed to the specific mechanism(s) responsible for constitutive NF-κB activity in each tumor sample, as the identity and relative abundance of active homo- and heterodimers depends on the nature of the activating signal or pathway aberrancy. Furthermore, as in human ABC-DLBCL patients, constitutive activity was associated with phosphorylation of IKKβ and IκBα in all but one dog evaluated, indicating that in the majority of cases signals leading to constitutive activity occur upstream of the IKK complex. These findings show on a molecular level that human ABC-DLBCL and canine DLBCL have similar aberrant NF-κB signaling and that the dog can serve as a clinically relevant large animal model.

Although constitutive NF-κB activity was present in the malignant lymph nodes of all dogs with untreated DLBCL, the NF-κB target genes Bcl-2, c-FLIP and XIAP were over-expressed in only half of the biopsy samples. These findings serve to underline the multiple layers of complexity involved in the regulation of NF-κB target gene expression. Firstly, the particular combination of activated NF-κB family members influences the target gene expression profile, in part because these family members have different binding affinities for different gene promoters. While NF-κB activation originates upstream of IKK in these dogs, the specific activation triggers have not been investigated and may differ between dogs, resulting in different NF-κB family member usage and different target gene expression signatures. These points highlight the complex and multifaceted nature of NF-κB regulated gene expression.

In the second part of this study we show that NBD peptide can inhibit constitutive IKKβ and IκBα phosphorylation in DLBCL biopsy samples which leads to increased apoptosis of malignant lymphocytes. Furthermore, NBD peptide showed a dose-dependent inhibition of NF-κB target gene expression in dogs with relapsed, refractory large B-cell lymphoma. In 2 dogs receiving the lower NBD peptide dose (dogs 1 and 3), all NF-κB target genes increased up to 4-fold 24 hours post NBD peptide showing that constitutive NF-κB activity in these dogs may be exerting a transcriptional repressive effect. No correlation was identified between 24-hour gene expression profile results and change in lymph node mass 10 days later.

In 3 out of 4 dogs, intra-nodal administration of NBD peptide led to a marked reduction in tumor mass when compared to the contra-lateral, malignant lymph node that did not receive NBD peptide. In dogs that received multi-node injections of NBD peptide, the apparent effects on tumor mass were variable. While most nodes continued to increase in size despite NBD treatment and rescue chemotherapy, several nodes in both dogs showed a marked reduction in tumor mass. In both cases, responding nodes were smaller than non-responsive nodes at the time of treatment. These results indicate that the NBD peptide dose and its ability to diffuse within malignant nodes can influence its ability to exert a measurable therapeutic effect.

Our finding shows that inhibitors of both IKKα and IKKβ activity (such as NBD peptide) will provide more complete inhibition and have greater therapeutic efficacy than IKKβ inhibitors alone. Furthermore, agents such as NBD peptide that specifically target the scaffolding molecule NEMO are less likely than kinase inhibitors to exhibit off-target effects on other intracellular signaling pathways.

In this study, NBD peptide did not cause any systemic toxicity and there were no significant changes in serum chemistries or hematological profiles over the 10 day assessment period that could be attributable to NBD peptide administration.

Taken together, the results reported here demonstrate that NBD peptide can inhibit anti-apoptotic target gene expression driven by constitutive NF-κB activity in vivo and that this inhibition, either alone or in combination with cytotoxic therapy, can provide improved clinical responses in dogs with ABC-like DLBCL. Given our findings that demonstrate the dog is a highly relevant, spontaneous clinical model for ABC-DLBCL in humans, the therapeutic effects of NF-κB inhibition seen in the canine cancer patient are likely to hold high translational relevance to human patients with ABC-DLBCL.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A method for treating an activated B-Cell Diffuse Large B-Cell Lymphoma (ABC-DLBCL) in a subject, the method comprising: administering to said subject a therapeutically effective amount of an NF-κB activation inhibitor, wherein said NF-κB activation inhibitor is an NF-κB Essential Modulator (NEMO) Binding Domain (NBD) peptide or a mimetic thereof, thereby treating said ABC-DLBCL in said subject.

2. The method of claim 1, wherein said NF-κB activation inhibitor is an agent that inhibits the interaction of both IKKα and IKKβ with NEMO and prevents the assembly of IKK complex.

3. The method of claim 1, wherein said NF-κB activation inhibitor is a molecule that is capable of diminishing the activity of NF-κB.

4. The method of claim 1, wherein said NF-κB activation inhibitor is co-administered with a chemotherapy agent.

9. The method of claim 1, said NF-κB activation inhibitor is administered independently from an administration of a chemotherapy agent.

10. The method of claim 1, wherein the administration of said NF-κB activation inhibitor inhibits the growth of a tumor associated with ABC-DLBCL.

11. The method of claim 1, wherein said subject is a human.

12. The method of claim 1, wherein said subject is a dog.

13. A method for inhibiting the growth of a tumor associated with a relapsed, refractory large B-Cell lymphoma in a subject, the method comprising: administering to said subject a therapeutically effective amount of an NF-κB activation inhibitor, wherein said an NF-κB activation inhibitor is an NF-κB Essential Modulator (NEMO) Binding Domain (NBD) peptide or a mimetic thereof, thereby inhibiting the growth of said tumor associated with said relapsed, refractory large B-Cell lymphoma in said subject.

14. The method of claim 13, wherein said NF-κB activation inhibitor is an agent that inhibits the interaction of both IKKα and IKKβ with NEMO and prevents the assembly of IKK complex.

15. The method of claim 13, wherein said NF-κB activation inhibitor is a molecule that is capable of diminishing the activity of NF-κB.

16. The method of claim 13, wherein said NF-κB activation inhibitor is co-administered with a chemotherapy agent.

17. The method of claim 13, said NF-κB activation inhibitor is administered independently from an administration of a chemotherapy agent.

18. The method of claim 13, wherein said subject is a human.

19. The method of claim 13, wherein said subject is a dog.

20. A pharmaceutical composition to treat an activated B-Cell Diffuse Large B-Cell Lymphoma (ABC-DLBCL) in a subject, comprising: a therapeutically effective amount of an NF-κB activation inhibitor, wherein said an NF-κB activation inhibitor is an NF-κB Essential Modulator (NEMO) Binding Domain (NBD) peptide or a mimetic thereof, wherein said NF-κB activation inhibitor is present in an amount effective to treat said ABC-DLBCL.

21. The composition of claim 20, further comprising a chemotherapy agent that treats said ABC-DLBCL.

22. The composition of claim 20, wherein said NF-κB activation inhibitor is present in an amount effective to enhance the efficacy of a chemotherapy agent.

23. The composition of claim 20, wherein said NF-κB activation inhibitor is an agent that inhibits the interaction of both IKKα and IKKβ with NEMO and prevents the assembly of IKK complex.

24. The composition of claim 20, wherein said NF-κB activation inhibitor is a molecule that is capable of diminishing the activity of NF-κB.

25. The composition of claim 20, wherein the administration of said NF-κB activation inhibitor inhibits the growth of a tumor associated with ABC-DLBCL.

26. The composition of claim 20, wherein said subject is a human.

27. The composition of claim 20, wherein said subject is a dog.

28. A pharmaceutical composition to inhibit the growth of a tumor associated with a relapsed, chemoresistant large B-Cell lymphoma in a subject, comprising: a therapeutically effective amount of an NF-κB activation inhibitor, wherein said NF-κB activation inhibitor is an NF-κB Essential Modulator (NEMO) Binding Domain (NBD) peptide or a mimetic thereof, and wherein said NF-κB activation inhibitor is present in an amount effective to inhibit the growth of said tumor associated with said relapsed, chemoresistant large B-Cell lymphoma.

29. A method for enhancing a response to chemotherapy to treat an activated B-Cell Diffuse Large B-Cell Lymphoma (ABC-DLBCL) in a subject, the method comprising: administering to said subject a therapeutically effective amount of an NF-κB activation inhibitor, wherein said NF-κB activation inhibitor is an NF-κB Essential Modulator (NEMO) Binding Domain (NBD) peptide or a mimetic thereof, thereby enhancing said response to chemotherapy to treat said ABC-DLBCL in said subject.

30. A method for treating an activated B-Cell Diffuse Large B-Cell Lymphoma (ABC-DLBCL) in a subject, the method comprising: collecting a biological sample from said subject; determining the level of NF-κB activity in said sample; administering to said subject a therapeutically effective amount of an NF-κB activation inhibitor if the level of NF-κB activity is higher than a standard pre-determined level, wherein said NF-κB activation inhibitor is an NF-κB Essential Modulator (NEMO) Binding Domain (NBD) peptide or a mimetic thereof, thereby treating said ABC-DLBCL in said subject.
